(19) United States
(12) Patent Application Publication
(10) Pub. No.: US 2025/0303163 A1
(43) Pub. Date: Oct. 2, 2025

(54) EXTREMELY LOW DUTY-CYCLE ACTIVATION OF THE CHOLINERGIC ANTI-INFLAMMATORY PATHWAY TO TREAT CHRONIC INFLAMMATION

(71) Applicant: SetPoint Medical Corporation, Valencia, CA (US)

(72) Inventors: Jacob A. LEVINE, West Hempstead, NY (US); Michael A. FALTYS, Valencia, CA (US)

(21) Appl. No.: 19/235,529

(22) Filed: Jun. 11, 2025

Related U.S. Application Data
(63) Continuation of application No. 15/853,350, filed on Dec. 22, 2017, now Pat. No. 12,357,826.
Application No. 15/853,350 is a continuation of application No. 14/968,702, filed on Dec. 14, 2015, now Pat. No. 9,849,286.
Application No. 14/968,702 is a continuation of application No. 14/336,942, filed on Jul. 21, 2014, now Pat. No. 9,211,410.
Application No. 14/336,942 is a continuation-in-part of application No. 13/467,928, filed on May 9, 2012, now Pat. No. 8,788,034.
(60) Provisional application No. 61/484,112, filed on May 9, 2011.

(51) Int. Cl. A61N 1/36; A61N 1/372

(52) U.S. Cl. CPC A61N 1/36053; A61N 1/36139; A61N 1/36167; A61N 1/36175; A61N 1/37205

(57) ABSTRACT

Described herein are systems and methods for applying extremely low duty-cycle stimulation sufficient to treat chronic inflammation using feedback to adjust the off times between stimulations. In particular, the feedback include an assessment of the level of inflammation by the patient or the healthcare provider, or by measure the level of an inflammatory analyte or biomarker, or by detecting nerve activity correlated with inflammation.

### CROSS REFERENCE TO RELATED APPLICATIONS

This application is a continuation of U.S. patent application Ser. No. 15/853,350, filed Dec. 22, 2017, titled “EXTREMELY LOW DUTY-CYCLE ACTIVATION OF THE CHOLINERGIC ANTI-INFLAMMATORY PATHWAY TO TREAT CHRONIC INFLAMMATION,” now U.S. Patent Application Publication No. 2018/0117320, which is a continuation of U.S. patent application Ser. No. 14/968,702, filed Dec. 14, 2015, titled “EXTREMELY LOW DUTY-CYCLE ACTIVATION OF THE CHOLINERGIC ANTI-INFLAMMATORY PATHWAY TO TREAT CHRONIC INFLAMMATION,” now U.S. Pat. No. 9,849,286, which is a continuation of U.S. patent application Ser. No. 14/336,942, filed Jul. 21, 2014, titled “EXTREMELY LOW DUTY-CYCLE ACTIVATION OF THE CHOLINERGIC ANTI-INFLAMMATORY PATHWAY TO TREAT CHRONIC INFLAMMATION,” now U.S. Pat. No. 9,211,410, which is a continuation-in-part of U.S. patent application Ser. No. 13/467,928, filed May 9, 2012, titled “SINGLE-PULSE ACTIVATION OF THE CHOLINERGIC ANTI-INFLAMMATORY PATHWAY TO TREAT CHRONIC INFLAMMATION,” now U.S. Pat. No. 8,788,034, which claims the benefit of U.S. Provisional Patent Application No. 61/484,112, filed May 9, 2011, and titled “SINGLE-PULSE ACTIVATION OF THE CHOLINERGIC ANTI-INFLAMMATORY PATHWAY TO TREAT CHRONIC INFLAMMATION,” each of which is hereby incorporated by reference in its entirety.

This patent application may be related to any of the following patent and pending patent applications: U.S. patent application Ser. No. 12/434,462, filed May 1, 2009, titled “VAGUS NERVE STIMULATION ELECTRODES AND METHODS OF USE,” Publication No. US-2009-0275997-A1; U.S. patent application Ser. No. 12/620,413, filed Nov. 17, 2009, entitled “DEVICES AND METHODS FOR OPTIMIZING ELECTRODE PLACEMENT FOR ANTI-INFLAMMATORY STIMULATION,” now U.S. Pat. No. 8,412,338; U.S. patent application Ser. No. 12/874,171, filed Sep. 1, 2010, titled “PRESCRIPTION PAD FOR TREATMENT OF INFLAMMATORY DISORDERS,” Publication No. US-2011-0054569-A1; U.S. patent application Ser. No. 12/917,197, filed Nov. 1, 2010, titled “MODULATION OF THE CHOLINERGIC ANTI-INFLAMMATORY PATHWAY TO TREAT PAIN OR ADDICTION,” Publication No. US-2011-0106208-A1; U.S. patent application Ser. No. 12/978,250, filed Dec. 23, 2010, titled “NEURAL STIMULATION DEVICES AND SYSTEMS FOR TREATMENT OF CHRONIC INFLAMMATION,” now U.S. Pat. No. 8,612,002; and U.S. patent application Ser. No. 12/797,452, filed Jun. 9, 2010 and entitled “NERVE CUFF WITH POCKET FOR LEADLESS STIMULATOR,” Publication No. US-2010-0312320-A1.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates generally to systems and devices for treatment of disorders, including chronic inflammation and inflammatory disorders using extremely low duty-cycle stimulation. In particular, described herein are systems, devices and methods for treating disorders such as intestinal inflammatory disorders. Further described herein generally are methods and devices, including an implantable microstimulators, adapted for electrically stimulating the vagus nerve to treat chronic inflammation by extremely low duty cycle stimulation to modulate an inflammatory response (via the nicotinic cholinergic anti-inflammatory pathway). In particular, described herein are systems and method adapted to increase the duration between stimulations (“off time”) while sustaining and even increasing the duration of equivalent inhibition.

### BACKGROUND

Electrical stimulation of the neural cholinergic anti-inflammatory pathway (CAP or NCAP) has been described in the literature, beginning with the seminal work of Kevin Tracey (see, e.g., Tracey, KJ “Physiology and immunology of the cholinergic anti-inflammatory pathway.” The Journal of clinical investigation 2007:117 (2): 289-96), who first identified the cholinergic anti-inflammatory pathway and characterized the link between vagus nerve stimulation and inhibition of inflammation by suppressing cytokine production. Since then, research as continued to explore the relationship between stimulation of the CAP and modulation of inflammatory disorders. Typical stimulation parameters have include stimulation by a burst of pulses (e.g., between 10 Hz to 1 GHz for between 30 sec and 20 min), with a slight increase in effect seen at higher frequencies (see, e.g., U.S. Publication No. 2009/0143831 to Huston et al.).

Although this work has suggested that chronic inflammation may be successfully treated by an implantable stimulator, the design and implementation of such a chronically implantable and usable stimulator has proven elusive, in part because of the power demands that a device capable of truly long-term, chronic, usage would face.

Implantable electrical stimulation devices have been developed for therapeutic treatment of a wide variety of diseases and disorders. For example, implantable cardioverter defibrillators (ICDs) have been used in the treatment of various cardiac conditions. Spinal cord stimulators (SCS), or dorsal column stimulators (DCS), have been used in the treatment of chronic pain disorders including failed back syndrome, complex regional pain syndrome, and peripheral neuropathy. Peripheral nerve stimulation (PNS) systems have been used in the treatment of chronic pain syndromes and other diseases and disorders. Functional electrical stimulation (FES) systems have been used to restore some functionality to otherwise paralyzed extremities in spinal cord injury patients.

Recently, implantable vagus nerve stimulations have been developed, including vagus nerve stimulators to treat inflammation. Such implants typically require an electrode and a power source. The size and use-limiting parameters may typically be the power requirements, which either require a long-lasting (and therefore typically large) battery, or require the added complication of charging circuitry and charging devices.

For example, typical implantable electrical stimulation systems may include one or more programmable electrodes on a lead that are connected to an implantable pulse generator (IPG) that contains a power source and stimulation circuitry. Even relatively small implantable neural stimulator technology, i.e. microstimulators, having integral electrodes attached to the body of a stimulator may share some of these disadvantages, as the currently developed leadless devices tend to be larger and more massive than desirable, making it difficult to stably position such devices in the proper position with respect to the nerve.

We herein describe the surprising result that long-lasting, robust inhibition of inflammation may be achieved by on a single (or very few) supra-threshold electrical pulse applied to the vague nerve. This finding is particularly surprising given the extraordinarily robust effect despite the minimal power applied, particularly compared to published data showing effects at much higher applied energy. These findings support various extremely low-power devices, system and methods for treating chronic inflammation. In particular, devices and methods for the treatment of inflammatory disorders, including inflammatory disorders of the intestine (e.g., irritable bowel disorder or IBD) are described, including microstimulators and methods of using them based on the remarkably low power requirements identified.

### SUMMARY OF THE DISCLOSURE

Described herein are devices, systems and methods for the extraordinarily low duty cycle stimulation of the vagus nerve. An extraordinarily low, extremely low, super low, or ultra low duty cycle refers generally to a duty cycle that provides stimulation using both a low number of electrical pulses per time period and a low stimulation intensity such that power requirements of the duty cycle are very low. The following are examples of various embodiments of extraordinarily low, extremely low, super low, or ultra low duty cycles. In some embodiments, the number of electrical pulses can be between 1 and 5, in one pulse increments, every 4 to 48 hours (or every 48-72 hours, or ever 2-4 days, or every 2-5 days, or every 2-10 days, or every 2-14 days or every 2-18 days, or every 2-20 days or every 2-25 days, etc.), including in 4 hour increments. In some embodiments, the stimulation intensity can be at a supra-threshold level that is capable of effecting the desired physiological response through the vagus nerves. In some embodiments, the supra-threshold level is between about 100 μA and 5000 μA, or between about 100 μA and 4000 μA, or between about 100 μA and 3000 μA, or between about 100 μA and 2000 μA. In some embodiments, the supra-threshold level is less than about 2000 μA, 3000 μA, 4000 μA or 5000 μA.

In some embodiments, the duty cycle is one supra-threshold pulse every 4 hours, with the pulse amplitude less than about 2000 μA. In some embodiments, the duty cycle is one pulse every 4 hours, with the pulse amplitude less than about 3000 μA. In some embodiments, the duty cycle is one pulse every 12 hours, with the pulse amplitude less than about 2000 μA. In some embodiments, the duty cycle is one pulse every 12 hours, with the pulse amplitude less than about 3000 μA. In some embodiments, the duty cycle is one pulse every 24 hours, with the pulse amplitude less than about 2000 μA. In some embodiments, the duty cycle is one pulse every 24 hours, with the pulse amplitude less than about 3000 μA. In some embodiments, the duty cycle is one pulse every 48 hours, with the pulse amplitude less than about 2000 μA. In some embodiments, the duty cycle is one pulse every 48 hours, with the pulse amplitude less than about 3000 μA.

In some embodiments the pulse width can be between about 100 to 1000 μS, or can be about or less than about 100, 200, 300, 400, 500, 600, 700, 800, 900 or 1000 μS. In some embodiments, the frequency can be about or less than about 10, 20, 30, 40, 50, 60, 70, 80, 90 or 100 Hz. In some embodiments, the IPI can be about or less than about 100, 200, 300, 400, 500, 600, 700, 800, 900 or 1000 μS.

In some embodiments, a system for treating chronic inflammation and/or an inflammatory disorder in a subject is provided. The system includes an implantable microstimulator configured to apply a low duty-cycle stimulation to a vagus nerve, wherein the low duty-cycle stimulation provides no more than a single supra-threshold pulse every four hours; and a controller configured to set a dose for the microstimulator wherein the dose comprises the single supra-threshold pulse followed by an off-period of at least four hours. In some embodiments, the off-period is at least 24 hours, or at least 48 hours, or between about 4 to 48 hours, or between about 12 to 48 hours, or between about 24 to 48 hours. In some embodiments, the single supra-threshold pulse has a pulse amplitude of less than 5 mA, less than 3 mA, or less than 2 mA. In some embodiments, the single supra-threshold pulse is biphasic. In some embodiments, the chronic inflammation is intestinal inflammation. In some embodiments, the chronic inflammation is inflammatory bowel disease. In some embodiments, the chronic inflammation is Crohn's disease.

In some embodiments, a method of treating chronic inflammation and/or inflammatory disorders in a subject is provided. The method includes implanting a microstimulator; and applying only a single supra-threshold stimulus pulse from the microstimulator to the vagus nerve followed by an off-time of at least 4 hours. In some embodiments, the off-time is at least 24 hours, at least 48 hours, or between about 4 to 48 hours, or between about 12 to 48 hours, or between about 24 to 48 hours. In some embodiments, the single supra-threshold stimulus pulse has a pulse amplitude of less than 5 mA, less than 3 mA, or less than 2 mA. In some embodiments, the single supra-threshold stimulus pulse is biphasic. In some embodiments, the chronic inflammation is intestinal inflammation. In some embodiments, the chronic inflammation is inflammatory bowel disease. In some embodiments, the chronic inflammation is Crohn's disease.

Types of inflammatory disorders that may be treated as described herein include a variety of disease states, including diseases such as hay fever, atherosclerosis, arthritis (rheumatoid, bursitis, gouty arthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, etc.

Non-limiting examples of inflammatory disorders which can be treated using the present invention include appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety and stent thrombosis.

The systems and methods described herein generally relate to systems and devices for treatment of chronic inflammation and inflammatory disorders. In particular, described herein are systems, devices and methods for treating intestinal disorders and rheumatoid arthritis. Further described herein generally are methods and devices, including an implantable microstimulators, adapted for electrically stimulating the vagus nerve to treat chronic inflammation by extremely- or super-low duty cycle stimulation and by extremely low treatment dose schedule to modulate an inflammatory response (via the cholinergic anti-inflammatory pathway).

For example, any of the systems and methods described herein may include or be specifically adapted and/or configured to deliver a treatment regimen in which the delay between stimulation doses (including single bursts and/or single pulses of supra-threshold stimulation) is progressively increased from the start of stimulation so that subsequent (later) stimulation occurs with longer off-times than earlier doses, without substantially decreasing the inhibition of inflammation due to the vagal stimulation. This effect may be referred to herein as ‘training’ the subject or vagus nerve, as the later stimulation (following an initial training period) may achieve the same or even more robust inhibition of inflammation with a longer duration between applied vagal stimulation. In general, the effect of VNS stimulation described herein may be referred to as an inhibition of the inflammatory response, and may include the inhibition of cytokines, or the increase of anti-inflammatory cytokines, or both.

For example, described herein are systems for treating chronic inflammation in a subject that include: an implantable microstimulator configured to apply a low duty-cycle stimulation to a vagus nerve; and a controller adapted to set a dose regimen of progressively delayed supra-threshold stimulus pulses for the microstimulator, wherein the dose regimen comprises a first dose comprising a supra-threshold stimulus pulse followed by a first off-period of at least about 48 hours, a second dose comprising a supra-threshold stimulation pulse followed by a second off-period that is longer than the first off-period, and a series of sequential doses each comprising a supra-threshold stimulation pulse followed by an off-period that is longer than the second off-period, wherein the supra-threshold stimulus pulses are configured to reduce a level of inflammation in the subject.

The first off-period is may be least about 72 hours, or 3.5 days, 4 days, 5 days, 6 days or 7 days, etc., and the second off period may be at least about 1.1 to 3 times the first off period (e.g., 5 days, 6 days, 7 days, 8 days, 9 days, 10 days, 11 days, 12 days, 13 days, 14 days, etc.). For example, the second and/or subsequent off-periods may be between about 1.1 and 2 times the first off period (e.g., 1.2 and 2.2 times, etc.). In one example, illustrated below, the first off-period is about 7 days and the second off period is at least about 10 days. In general, the off-period is the quiescent period during which no supra-threshold (and/or no stimulation at all) is applied by the implant to the vagus. In general, the time of the first off period may be determined based on the amount of inhibition of inflammation. For example, the duration of the first off period and subsequent off periods may be determined by examining the level of inhibition of inflammation (of an inflammatory response) or of a marker for inflammation and/or the inflammatory response. For example, the off-period may extend until inflammation or a marker for inflammation and/or the inflammatory response (either ongoing or evoked from the subject) is a percentage of the native inflammation level or inflammatory response (e.g., above about 30%, 40%, 50%, 60%, 70%, 75%, 80%, 85%, 90%, 95%), etc. Thus, for example, the off-periods of the sequential doses may each at least about two weeks. In some variations, the off-periods of the sequential doses are ramped up to a predetermined length of time. Thus, for example, the second off-period may be longer than the first off-period and subsequent off-periods may be longer still (progressively longer), but may approach a limit (e.g., of two weeks, 18 days, 3 weeks, 25 days, etc.). The limit may be a maximum delay period. In general, in the methods and apparatuses described herein, the application of the ultra-low duty-cycle stimulation at the progressive off-times described herein may result in a tonic (ongoing) inhibition of inflammation (or of markers for inflammation) at an acceptable level. This may permit remarkably low-power (or low-power consumption) devices that may be operated for many days, weeks or even months, without requiring power replacement or recharging while maintaining efficacy.

As mentioned above, the first dose may comprise a single supra-threshold stimulus pulse, or burst of pulses. A burst of pulses typically has a burst duration of less than about 5 minutes, less than about 4 min, 3 min, 2 min, 1 min, etc.). One or more (including all) of the pulses in the burst may be supra threshold. In some variations, for example, the dose (including the first dose) comprises a single burst of supra-threshold stimulus pulses.

Any of the devices and methods described herein may be configured to sense an indicator of the subject's inflammation or inflammatory response. For example, a system may include an analyte detector configured to measure a level of an inflammatory analyte in the subject's blood or bodily fluids (e.g., the level of a marker of an inflammatory response). Some variations may include a sensor configured to detect a measure of inflammation based on the electrical activity of the vagus nerve; thus any of these systems may include one or more electrodes to sense activity on the vagus nerve. In any of these systems, the system (e.g., the controller) may be configured to adjust the doses (e.g., the second dose and/or subsequent doses) based on the level of inflammation in the subject, e.g., the level of inhibition of inflammation in the subject. For example, a controller may be configured to adjust the second dose and subsequent doses based on the level of an inflammatory analyte, and/or the controller may be configured to adjust the second off-period based on the level of inflammation in the subject (e.g. the level of the inhibition of the inflammatory response).

The controller may be configured to adjust the second off period based on the level an inflammatory analyte, e.g., based on the amount of inhibition of the inflammatory response. As mentioned, the microstimulator may include a sensing electrode configured to monitor vagus nerve activity; this activity may be analyzed (e.g., by the microstimulator or remotely from the microstimulator, which may transmit and receive data and/or command information or instructions). The microstimulator may comprise a sensing electrode configured to monitor vagus nerve activity, and also a processor configured to process the monitored vagus nerve activity to determine a level of inflammation and/or the level of inhibition of inflammation.

Also described herein are methods of treating chronic inflammation in a patient by progressively increasing the off-times between stimulation. For example, a method may include: applying a single supra-threshold stimulus from a microstimulator to a vagus nerve, wherein the delivery of the stimulus is followed by a first off-time of at least about 48 hours during which an inflammatory response is suppressed; and applying subsequent supra-threshold stimuli, wherein each subsequent stimulus is followed by an off-time of longer than 48 hours.

The step of applying the single supra-threshold stimulus may include applying a single burst of pulses, or a single supra-threshold pulse. As mentioned above, the off-times may be predetermined as part of the dosing regimen (e.g., the first off-time may at least about 72 hours, 4 days, 5 days, 6 days, 7 days, etc.). The first off-time may be, for example, at least about 7 days. The subsequent off-times may be predetermined and/or may be modified by one or more subject-specific parameters, including, for example, the level of inhibition of the inflammatory response for the subject. For example, after the first or second stimulation doses are applied, the subsequent off-times may be at least about one to two weeks. As mentioned, the subsequent off times may be ramped up from the first off-time to a longer predetermined length of time (e.g., up to two weeks, 2.5 weeks, three weeks, 3.5 weeks, four weeks, etc.).

In any of these variations, the method may include a step of determining the level of inflammation (or the level of inhibition of the inflammatory response) and adjusting the off-times following the subsequent supra-threshold stimuli based on the level of inflammation and/or the level of inhibition of the inflammatory response. For example, the level of inflammation and/or inhibition of inflammation may be estimated by monitoring vagus nerve activity; the off-times following the subsequent supra-threshold stimuli may be adjusted based on the level of inflammation and/or the level of inhibition of inflammation.

In general, any of these methods may also include determining the level of an inflammatory analyte in the subject's blood or bodily fluids and adjusting the off-times following the subsequent supra-threshold stimuli based on the level of analyte. The level of the analyte may be indicative of the level of inflammation and/or the level of inhibition of inflammation. For example, a level of inhibition of inflammation may be determined by comparison to a baseline (e.g., prior to vagus nerve stimulation as described). The level of inhibition of inflammation may be determined as a percentage of inhibition of this inflammatory response. The inflammatory response may be determined by evoking (e.g., ex vivo or in vivo) an inflammatory response and comparing it to a current (or some post-stimulation) time point.

Also described herein are methods of treating chronic inflammation in a subject by progressively increasing the off-times between stimulation. For example, a method may comprise: applying to a vagus nerve from an implanted microstimulator, a first dose comprising a supra-threshold stimulus, followed by a first off-time of at least about 48 hours, wherein the application of the first dose reduces the level of inflammation in the subject; applying a second dose comprising a supra-threshold stimulus, followed by a second off-time that is longer than the first off-time; and applying subsequent doses comprising supra-threshold stimuli, wherein each does is followed by an off-time that is longer than the second off-time.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a diagram of a single stimulation waveform;

FIG. 2 is a graph comparing the effect on TNF levels from a single stimulation pulse with the effect from up to 3000 pulses;

FIG. 3 is a graph illustrating the effect on TNF levels from a single stimulation pulse 24 hours post-stimulation;

FIG. 4 is a graph illustrating the effect on TNF levels from a single stimulation pulse 3 hours and 24 hours post-stimulation;

FIG. 5 is a graph illustrating the effect on lesion area in a rat model of IBD from a single stimulation pulse;

FIG. 6 is a graph illustrating the long term effect on lesion area in a rat model of IBD from a single stimulation pulse;

FIG. 7 is a diagram of an embodiment of a stimulation waveform;

FIGS. 8A and 8B illustrate an embodiment of a nerve cuff lead that has been implanted around the vagus nerve;

FIG. 8C illustrates an embodiment of a microstimulator; and

FIG. 9 presents canine data that shows that vagus nerve stimulation can achieve a long lasting anti-inflammatory effect and that the longevity of the effect can be increased.

FIG. 10 is another example of vagus nerve stimulation with progressively longer off-times between extremely low duty-cycle does from a microstimulator on the vagus nerve. The percent inhibition of inflammation may be determined from the level of an analyte in the blood and/or vagus nerve activity, as described herein.

### DETAILED DESCRIPTION

In general, described herein are systems, methods and devices illustrating extraordinarily low duty cycle stimulation of the vagus nerve to treat a disorder. In particular, described herein are systems, methods and devices illustrating extraordinarily low duty cycle stimulation of the vagus nerve to reduce or prevent inflammation and the effects of inflammation in a mammalian model. An extraordinarily low, extremely low, super low, or ultra low duty cycle refers generally to a duty cycle that provides stimulation using both a low number of electrical pulses per time period and a low stimulation intensity such that power requirements of the duty cycle are very low. The methods described herein apply various stimulation protocols that may be used to significantly reduce inflammation and/or the effects of inflammation. Simulation parameters that may be varied include the pulse shape (e.g., sinusoidal, square, biphasic, monophasic, etc.) the duration of stimulation, the on-time, the off-time, the inter-pulse interval, or the like. One key factor examined herein is the number of supra-threshold pulses. As shown herein, the stimulation of the vagus nerve with even a single supra-threshold stimulus results in a significant and long-lasting effect, even when compared to multiple stimulations. This effect was particularly profound when examined using a rodent model for IBD.

The following are examples of various embodiments of extraordinarily low, extremely low, super low, or ultra low duty cycles. In some embodiments, the number of electrical pulses can be between 1 and 5, in one pulse increments, every 4 to 48 hours, in 4 hour increments. In some embodiments, the stimulation intensity can be at a supra-threshold level that is capable of effecting the desired physiological response through the vagus nerves. In some embodiments, the supra-threshold level is between about 100 μA and 5000 μA, or between about 100 μA and 4000 μA, or between about 100 μA and 3000 μA, or between about 100 μA and 2000 μA. In some embodiments, the supra-threshold level is less than about 2000 μA, 3000 μA, 4000 μA or 5000 μA.

In some embodiments, the duty cycle is one supra-threshold pulse every 4 hours, with the pulse amplitude less than about 2000 μA. In some embodiments, the duty cycle is one pulse every 4 hours, with the pulse amplitude less than about 3000 μA. In some embodiments, the duty cycle is one pulse every 12 hours, with the pulse amplitude less than about 2000 μA. In some embodiments, the duty cycle is one pulse every 12 hours, with the pulse amplitude less than about 3000 μA. In some embodiments, the duty cycle is one pulse every 24 hours, with the pulse amplitude less than about 2000 μA. In some embodiments, the duty cycle is one pulse every 24 hours, with the pulse amplitude less than about 3000 μA. In some embodiments, the duty cycle is one pulse every 48 hours, with the pulse amplitude less than about 2000 μA. In some embodiments, the duty cycle is one pulse every 48 hours, with the pulse amplitude less than about 3000 μA.

The examples described herein use a stimulator and stimulation control package that was developed for use in driving vagus nerve stimulation. In some example, the stimulation is controlled by a software package that is configured to run on a microprocessor (e.g., personal computer) and to control output of an emulator/stimulator (which may be referred to as an “ITE” or integrated terminal emulator). Thus, the systems described herein may include logic (e.g., control logic) that may be software, firmware, and/or hardware to control the application of stimulation. For example, in some variations, the parameters controlling stimulation and data acquisition may include: (1) selected stimulating electrode pair including a cathode and anode; (2) frequency in 1 Hz increments; (3) Pulse Width (PW): 20-2,000 uS in 1 uS increments; (4) Pulse Amplitude (PA): ±0-5,000 uA in 3 uA increments; and (5) Inter-Pulse-Interval between phase A & B of waveform (IPI): 20-2,000 uS in 1 uS increments.

In addition to the exemplary parameters provided above, in some embodiments the PW can be between about 100 to 1000 μS, or can be about or less than about 100, 200, 300, 400, 500, 600, 700, 800, 900 or 1000 μS. In some embodiments, the frequency can be about or less than about 10, 20, 30, 40, 50, 60, 70, 80, 90 or 100 Hz. In some embodiments, the IPI can be about or less than about 100, 200, 300, 400, 500, 600, 700, 800, 900 or 1000 μS.

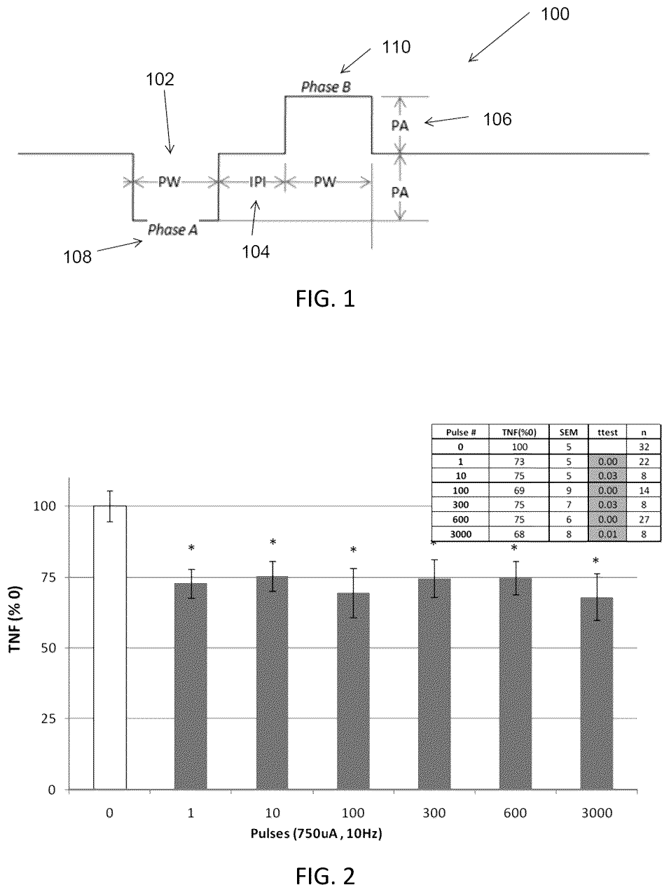

For example, the exemplary waveform shown on FIG. 1 is a biphasic (charge balanced) waveform 100 that includes two symmetric pulse widths 102 (PW) separated by an inter-pulse interval 104 (IPI). The pulse widths 102 have a pulse amplitude 106 (PA) that is also symmetric for the first phase 108 (phase A) and the second phase 110 (phase B) of the biphasic stimulus, with a negative pulse amplitude in phase A and a positive amplitude in phase B. This biphasic pulse is a single pulse that includes both a positive and negative excursion. Other pulse waveforms may be used. In some embodiments, the pulse waveforms may be non-biphasic and/or may have asymmetric pulse widths and/or asymmetric pulse amplitudes.

The stimulator may generate a pulse train on a pair of electrodes. In general, a does may include a single pulse (e.g., a single biphasic pulse) or a single burst including multiple pulses. The pulses may be generated using a bipolar current source and can be capacitively isolated with >1 uF ceramic capacitors on both electrodes outputs. Compliance voltage can be set to as high as +/−18.8 volts.

The different experimental examples described herein show that appropriate NCAP stimulation of the vagus nerve can be used to limit or eliminate the effects of intestinal inflammation, in particular in a rat model of colitis and a rat model of Crohn's disorder. Based on this data, a biphasic simulation at the parameters described above may successfully treat intestinal inflammation in humans or other mammals.

In one example, mice (Male, BALB/c) were anesthetized and cuff electrodes (0.3 mm ID, 0.5 mm inter-electrode distance; Microprobes, Gaithersburg, MD) were placed around the left carotid sheath (containing the cervical vagus nerve) and secured by suture. Supra-threshold pulses (750 μA, 200 μS, 10 Hz) were applied in various numbers (0, 1, 10, 100, 300, 600, 3000). Afterwards, the electrode was removed and the wound stapled closed. Mice recovered for 3 hours, and then were challenged with LPS (5 mg/kg; IP); these mice were sacrificed 90 minutes post-LPS and serum TNF measured by ELISA to measure the effects on inflammatory cytokines. As shown on FIG. 2, even a single supra-threshold stimulus resulted in a significant suppression of TNF at 3 hours after treatment. Thus, the effect was long lasting and the effect from a single pulse at 3 hours was equivalent to the effect generated by up to 3000 pulses.

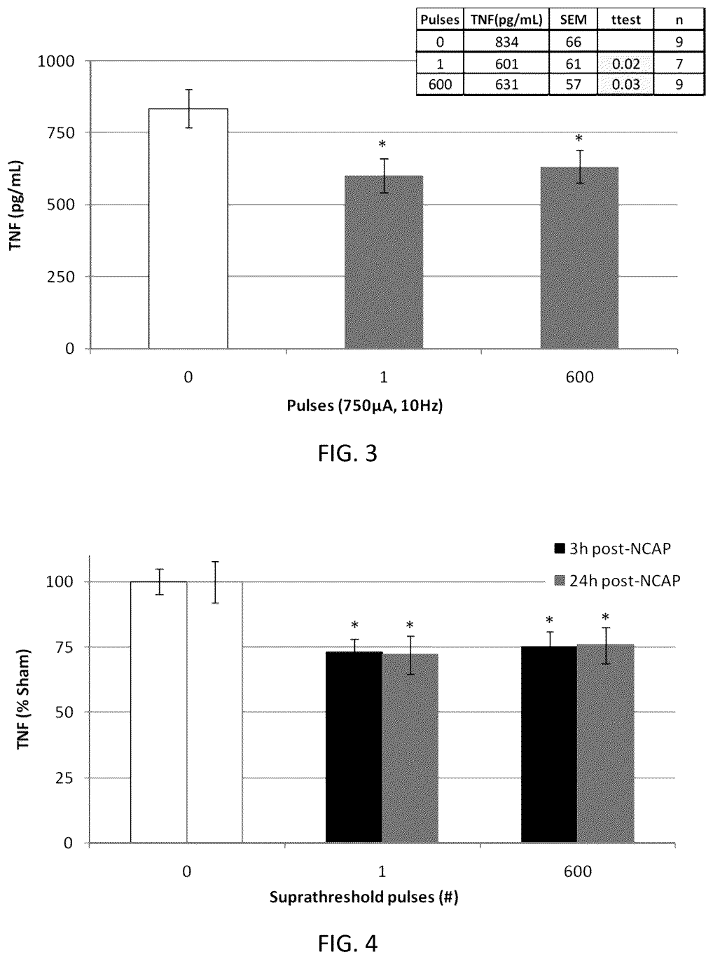

A second similar experiment was conducted to examine the long lasting effect of a single supra-threshold pulse on the cholinergic anti-inflammatory pathway (CAP). Mice (Male, BALB/c) were anesthetized and cuff electrodes (0.3 mm ID, 0.5 mm inter-electrode distance; Microprobes, Gaithersburg, MD) were placed around the left carotid sheath (containing the cervical vagus nerve) and secured by suture. Supra-threshold pulses (750 μA, 200 μS, 10 Hz) were applied in various numbers (0, 1, 600). Afterwards, the electrode was removed and the wound stapled closed. Mice recovered for 24 hours, and then were challenged with LPS (5 mg/kg; IP); these mice were sacrificed 90 minutes post-LPS and serum TNF measured by ELISA to measure the effects on inflammatory cytokines. As shown on FIG. 3, a single supra-threshold stimulus resulted in a significant suppression of TNF at 24 hours after treatment that was equivalent to the effect generated by 600 pulses.

FIG. 4 combines selected portions of the results of the two experiments described above to show that single pulse stimulation of the NCAP effects suppression of LPS-inducible TNF at 3 hours and 24 hours post-stimulation at the same effectiveness as 600 pulses.

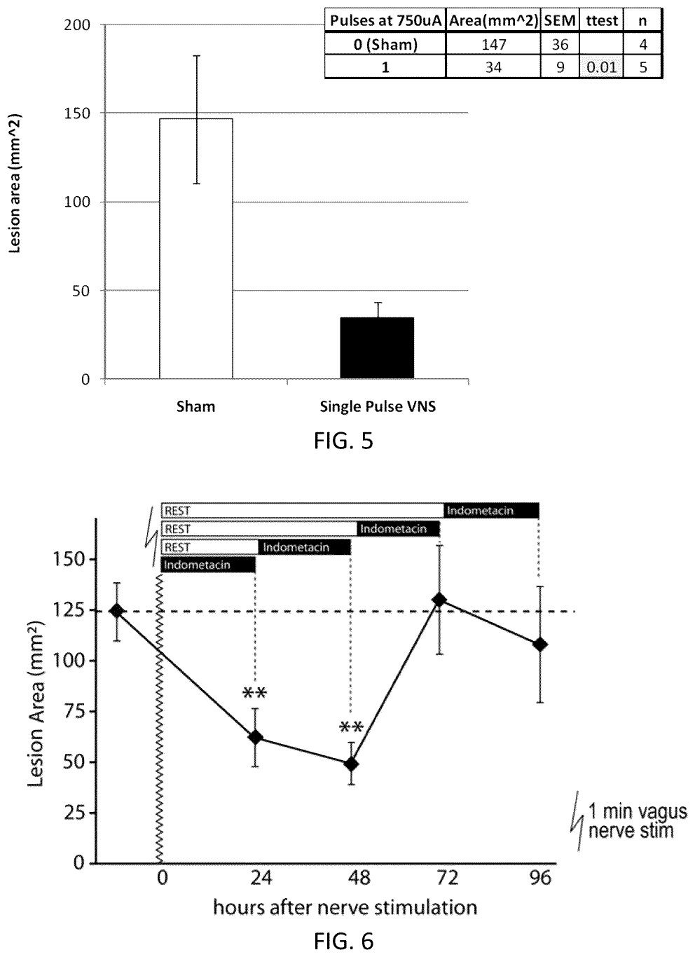

In another example, an experiment was conducted to determine the effectiveness of single pulse suppression of lesion area in a rat model for IBD/Crohn's disease. Rats were anesthetized and were either given a sham stimulation or a single supra-threshold stimulus to the left cervical vagus nerve (1 pulse at 750 μA, 200 μS pulse width, 10 Hz). IBD was induced at 30 minutes post-stimulation by the SC injection of indomethacin (10 mg/kg (5 mg/mL) in 5% sodium bicarbonate). Lesions were stained in-life 23.5 hours post-indomethacin injection by anesthetizing the rats with isoflurane and IV tail injection with Evans Blue (0.3 ml of 1%). Rats were sacrificed via C02 asphyxiation at 24 hours post disease induction, and the small intestines were harvested, cleaned and fixed in 2% formalin overnight. Photographs were taken and digitized of the fixed intestines and lesions were quantified by a blinded scorer. As illustrated in FIG. 5, a single supra-threshold stimulus (750 μA, 200 μS pulse width, 10 Hz) resulted in a profound reduction in lesions.

These results are even more significant, given the data shown in FIG. 6, which illustrates a “memory effect” of vagus nerve stimulation in a rat model of Crohn's disease. Rats were anesthetized and were either given a sham stimulation or an actual stimulation to the left cervical vagus nerve (1 mA, 200 μS pulse width, 10 Hz, 60 s). IBD/Crohn's disease was induced at various times (see FIG. 6) post-stimulation by the SC injection of indomethacin (10 mg/kg (5 mg/mL) in 5% sodium bicarbonate). Lesions were stained in-life 23.5 hours post-disease induction by anesthetizing the rats with isoflurane and IV tail injection with Evans Blue (0.3 ml of 1%). Rats were sacrificed via C0₂ asphyxiation at 24 hours post disease induction, and the small intestines were harvested, cleaned and fixed in 2% formalin overnight. Photographs were taken and digitized of the fixed intestines and lesions were quantified by a blinded scorer. In this example, a brief period of stimulation of the vagus nerve may result in a surprisingly long-lasting effect (e.g., up to 48 hours) in the reduction of intestinal lesions otherwise induced by the application of indomethacin. This data strongly suggests that stimulation may be provided extremely infrequently, with long (e.g., >48 hours) of “silent” periods without stimulation applied. Such extremely low duty-cycle stimulation for treating IBD may be particularly helpful in implantable systems, allowing extremely long battery life while having unexpectedly robust therapeutic benefits.

Although the examples provided above describe methods, systems and devices for treating an inflammatory disorder in a rat model, all the methods, systems and devices described herein can be used and/or adapted for use in other mammals, such as humans. For example, a system and method for treating an inflammatory disorder in a human using a single supra-threshold pulse and/or an extraordinarily low duty cycle stimulation protocol can include an electrode, such as a cuff electrode, that is configured to be implanted around the vagus nerve and deliver electrical stimulation to the vagus nerve of the subject. The system can further include a processor, memory for storing instructions, and/or a controller can include programming to deliver the low duty cycle stimulation protocol, including the single supra-threshold pulse protocol, to the vagus nerve via the cuff electrode. A battery can be provided to provide power for the system, and because the low duty cycle stimulation protocol consumes so little energy, the battery life can be greatly extended, allowing the system to be completely implanted within the subject for a long duration before the battery needs to be replaced or recharged. For an implanted system, this provides a great benefit since it can reduce the frequency of surgical procedures that may be required to change the battery.

The stimulation parameters used in this system can be the same or similar to the parameters disclosed above. For example, the pulse amplitude can be less than about 5, 4, 3, or 2 mA. In addition, the low duty cycle stimulation protocol can deliver a single supra-threshold pulse between off-times of between about 4 to 48 hours, or at least 4, 12, 24, or 48 hours. In some embodiments the pulse width can be between about 100 to 1000 μS, or can be about or less than about 100, 200, 300, 400, 500, 600, 700, 800, 900 or 1000 μS. In some embodiments, the frequency can be about or less than about 10, 20, 30, 40, 50, 60, 70, 80, 90 or 100 Hz. In some embodiments, the IPI can be about or less than about 100, 200, 300, 400, 500, 600, 700, 800, 900 or 1000 μS.

In general, these results suggest that the application of even a single brief supra-threshold stimulus of the vagus nerve may result in a substantial reduction in the effects of inflammation, possibly by inhibition of inflammatory cytokines such as TNF. These results are both surprising, given the prior arts tendency to stimulate for much longer times, and important for the design of future devices and methods. In particular, stimulation of the vagus nerve (or other portions of the inflammatory reflex) may be configured to apply extremely low duty-cycle stimulation. As mentioned briefly, this would allow for much smaller, lighter and more efficient implantable stimulation systems.

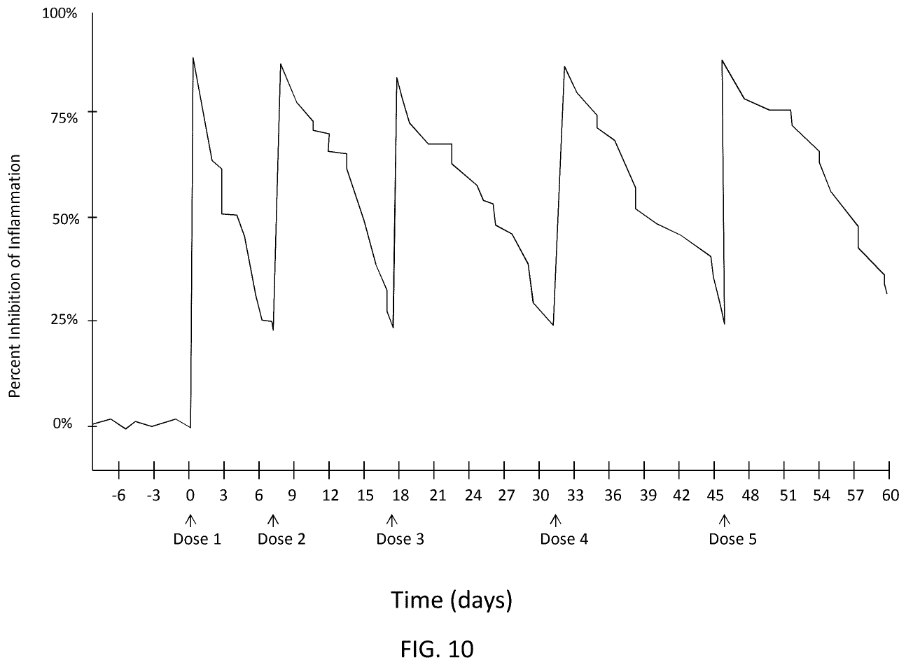

Another phenomenon seen in the experiments of FIGS. 2-7 is the training effect in which subsequent vagus nerve stimulation, particularly with extremely low duty-cycle stimulation, results in increasing the duration of suppression of the inflammatory response in the patient. For example, in mice, as well as other mammals into which a microstimulator has been applied to the vagus nerve, the first application of a dose of a dosing regimen (and particularly an extremely low duty-cycle dose), e.g., a since burst of supra-threshold pulses, e.g., having a burst duration of less than 5 min, 2 min, 1 min, etc., or even a single supra-threshold pulse, results in an inhibition of inflammation that lasts for many hours, and even days, as shown in FIG. 6. A second dose (e.g., pulse of supra-threshold stimulation) that is equivalent to the first pulse results in a longer-lasting inhibition than the first dose. Subsequent stimulation may also result in longer-lasting inhibition than preceding simulation. This means that the off-period between stimulation may be increased with subsequent stimulation, as is seen, for example, in FIGS. 9 and 10, discussed below. Preliminary data suggests that the lower duty-cycle (e.g., single pulse/single burst of limited burst duration) stimulation may be most effective in creating this enhanced duration of inhibition. When multiple (e.g., greater than 100 supra-threshold pulses, greater than 90 supra-threshold pulses, greater than 80 supra-threshold pulses, greater than 70 supra-threshold pulses, greater than 60 supra-threshold pulses, greater than 50 supra-threshold pulses, greater than 40 supra-threshold pulses, greater than 30 supra-threshold pulses, greater than 25 supra-threshold pulses, greater than 20 supra-threshold pulses, etc.) are used, significant enhancement of the duration of inhibition may not be robustly observed. Thus, it may be beneficial to limit the number of pulses in dose to less than 100, 90, 80, 70, 60, 50, 40, 30, 25, 20 etc. supra-threshold pulses, separated by an off-time of greater than 48 hours, 3 days, 4, days, etc.

Types of inflammatory disorders that may be treated as described herein include a variety of disease states, including diseases such as hay fever, atherosclerosis, arthritis (rheumatoid, bursitis, gouty arthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, etc.

Non-limiting examples of inflammatory disorders which can be treated using the present invention include appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety and stent thrombosis.

Any of these disorders (e.g., inflammatory disorders) may be treated by, for example, implanting a cuff electrode around the vagus nerve, and using an extraordinarily low duty cycle stimulation protocol as described herein to treat. A processor and memory for storing instructions and/or programming can be used to control the stimulation protocol. The stimulation parameters used in this system and method can be the same or similar to the parameters disclosed above. For example, the pulse amplitude of the single supra-threshold pulse can be less than about 5, 4, 3, or 2 mA. In addition, the low duty cycle stimulation protocol can deliver a single supra-threshold pulse between off-times of between about 4 to 48 hours, or at least 4, 12, 24, or 48 hours. Any of these methods may include a step of determining the efficacy of the treatment. For example, any of these methods may include the step of monitoring the subject before and/or during treatment. For example, in treating an inflammatory disorder, a biomarker for inflammation may be monitored, such as a cytokine or other marker. In some variations, monitoring the subject may include assessing the subject visually (e.g., for swelling, body temperature, etc.). In some variations the systems described herein may include a sensor and/or data processing subsystem for monitoring the subject and/or the effect of the treatment with the system.

Although the examples and description above focuses primarily on inflammatory disorders, in some embodiment, the systems, devices and methods described herein can be used to treat non-inflammatory diseases or disorders. For example, the systems, devices and methods described herein can be used to activate, regulate, and/or modulate the levels of sirtuins by extraordinarily low duty cycle stimulation of the vagus nerve. The modulation of sirtuins by vagus nerve stimulation is also discussed in U.S. patent application Ser. No. 13/338,185, filed Dec. 27, 2011, titled “MODULATION OF SIRTUINS BY VAGUS NERVE STIMULATION,” Publication No. US-2013-0079834-A1 which is hereby incorporated by reference in its entirety for all purposes. As above, a cuff electrode can be implanted around the vagus nerve and a processor and memory for storing instructions and/or programming can be used to control the stimulation protocol. The stimulation parameters used in this system and method can be the same or similar to the parameters disclosed above. For example, the pulse amplitude of the single supra-threshold pulse can be less than about 5, 4, 3, or 2 mA. In addition, the low duty cycle stimulation protocol can deliver a single supra-threshold pulse between off-times of between about 4 to 48 hours, or at least 4, 12, 24, or 48 hours. In some embodiments the pulse width can be between about 100 to 1000 μS, or can be about or less than about 100, 200, 300, 400, 500, 600, 700, 800, 900 or 1000 μS. In some embodiments, the frequency can be about or less than about 10, 20, 30, 40, 50, 60, 70, 80, 90 or 100 Hz. In some embodiments, the IPI can be about or less than about 100, 200, 300, 400, 500, 600, 700, 800, 900 or 1000 μS.

As mentioned above, in some embodiments, the system, device, and/or method includes monitoring the effects of the stimulation on the disease being treated. For example, inflammation indicators or disease indicators or other indicators can be monitored to evaluate the efficacy of the treatment protocol, allowing the stimulation protocol to be adjusted based on the evaluation. Any one of the parameters described herein can be modulated based on the evaluation. For example, the pulse amplitude and/or the off time can be increased or decreased to optimize the treatment efficacy. Examples of indicators that can be monitored include TNF levels, lesion size, degree or level of inflammation, cytokine levels, pain levels, sirtuin levels, and the like.

Another key factor examined herein is the longevity of effect of a single stimulation as well as the increase in the longevity of effect following a second stimulation that is applied after, for example, seven days later. In other embodiments, the second stimulation is delivered between 1-14 days after the first stimulation. In some embodiments, a third stimulation can be delivered 1-30 days after the second stimulation. More generally, the time period between stimulations can be increased after each stimulation until a desired or predetermined period of time between stimulations is achieved. In some embodiments, the time period between stimulations can be predetermined. The predetermined time period between stimulations may be constant, or can increase over time to a predetermined duration. In embodiments where the time period between stimulations is increased over time, the increase can be gradual, stepwise, or based according to a predetermined schedule. For example, the time period can be increased by 5, 10, 15, 20, or 25 percent, or by about 5-25 percent over the previous time period.

In other embodiments, the time period can be based on a measurement of analyte levels, biomarker levels, an assessment of the level of inflammation, and/or level or pattern or signature of vagus nerve activity, such that the next stimulation is applied when the analyte level, biomarker level, assessment of the level of inflammation, and/or level or pattern or signature of vagus nerve activity either exceeds or falls below a predetermined threshold. The levels or presence of analytes and biomarkers can also be indicators for inflammation. For example, the analyte can be TNF or another inflammatory cytokine or mediator. In some embodiments, the inflammatory analyte or biomarker can be measured ex vivo in a whole blood response assay or in another assay using whole blood or blood plasma. The assessment of the level of inflammation can also be a clinical assessment and/or a patient assessment, and can include a measurement and/or scoring of swelling and/or pain. In some embodiments, the measurements and/or assessments can be performed at predetermined intervals, such as daily, or every two days, which can begin immediately or after a predetermined time has elapsed, such as after 1, 2, 3, 4, 5, 6, or 7 days, for example. The level or pattern or signature of vagus nerve activity can also be correlated with levels of inflammation, allowing level of inflammation to be determined by monitoring of vagus nerve electrical activity, which can be done with a microstimulator with electrical sensing leads and signal processing circuitry and/or software, which can be on the microstimulator or on a computing device in communication with the microstimulator. In some embodiments, various combinations of the above can be used to increase the time period between stimulations.

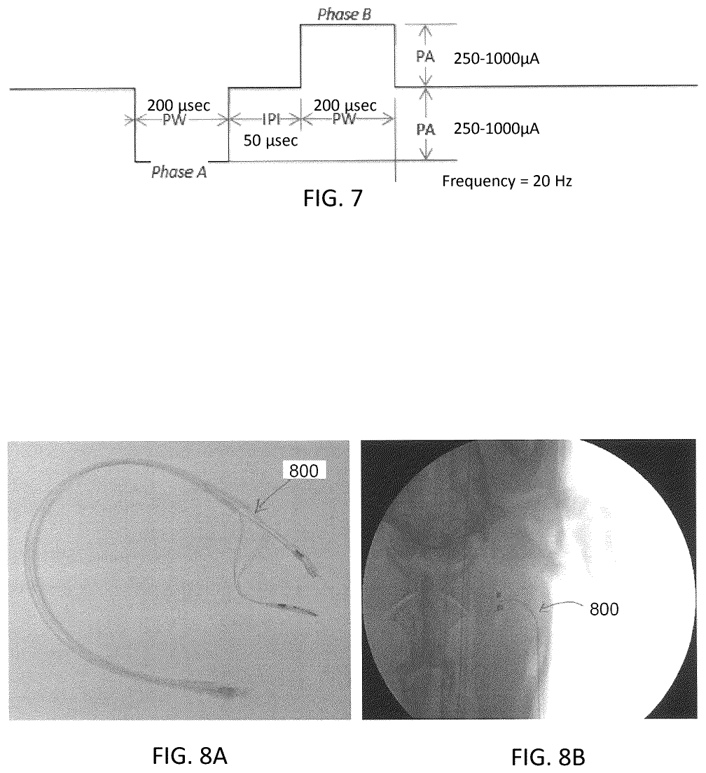

The examples described herein may use a stimulator and stimulation control package that was developed for use in driving vagus nerve stimulation. In some examples, the stimulation is controlled by a software package that is configured to run on a microprocessor (e.g., personal computer) and to control output of an emulator/stimulator (which may be referred to as an “ITE” emulator stimulator). With reference to FIG. 7, the parameters controlling stimulation and data acquisition may include: (1) selected stimulating electrode pair including a cathode and Anode; (2) frequency in 1 Hz increments; (3) Pulse Width (PW): 20-2,000 uS in 1 uS increments; (4) Pulse Amplitude (PA): ±0-5,000 uA in 3 uA increments; and (5) Inter-Pulse-Interval between phase A & B of waveform (IPI): 20-2,000 uS in 1 uS increments.

For example, the exemplary waveform shown on FIG. 7 is a biphasic (charge balanced) waveform that includes two symmetric pulse widths (PW, one positive, one negative) separated by an inter-pulse interval (IPI). The pulse widths have a pulse amplitude (PA) that is also symmetric for the first phase (phase A) and the second phase (phase B) of the biphasic stimulus. Other pulse waveforms may be used. Exemplary parameters for a waveform are shown in FIG. 7, which illustrates a waveform used in an experiment described in more detail below, where the waveform had a pulse width of 200 μsec, an inter-pulse-interval of 50 μsec, a pulse amplitude of 250-1000 μA, and a frequency of 20 Hz.

The stimulator may generate a pulse train on a pair of electrodes. The pulses may be generated using a bipolar current source and can be capacitively isolated with >1 uF ceramic capacitors on both electrodes outputs. Compliance voltage can be set to as high as +/−18.8 volts.

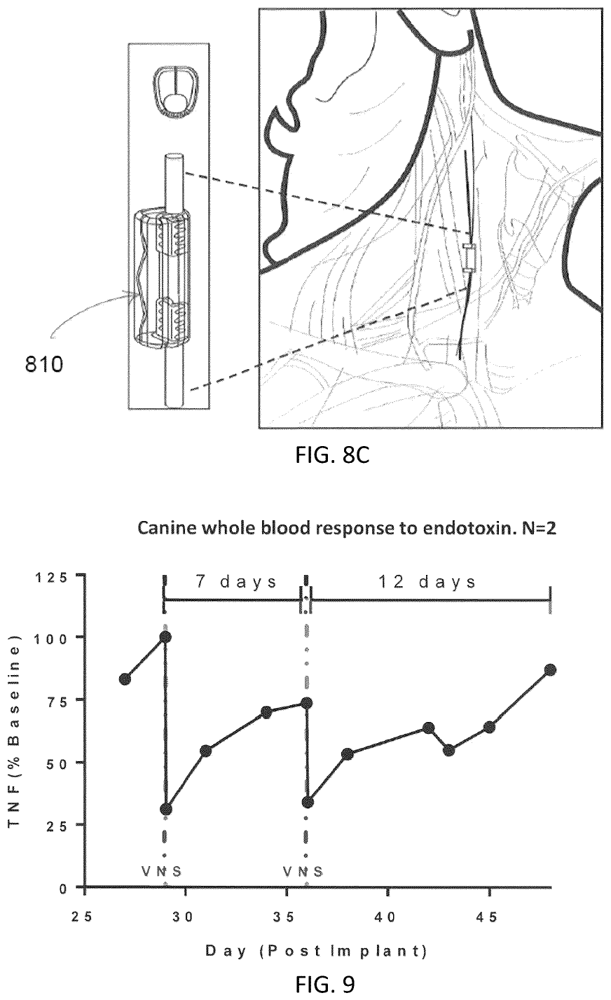

The stimulator may use traditional electrode configurations, such as a cuff electrode 800 illustrated in FIG. 8A. Alternatively, the stimulator may be a microstimulator 810 as illustrated in FIG. 8C, which is further described in U.S. Pat. No. 8,612,002, which is herein incorporated by reference in its entirety.

As illustrated in the experimental example described below, appropriate NCAP stimulation of the vagus nerve can be used to limit the TNF inducibility of leukocytes in ex vivo blood by endotoxin, a reflection of the inflammatory responsiveness of the subject. Based on this data, a biphasic simulation at the parameters described above may successfully treat inflammatory disease, with progressively longer duration of anti-inflammatory effect with each successive stimulation.

FIGS. 8A and 8B illustrate one example in which progressively longer off-times were used to achieve a sustained inhibition of inflammation. Two canines (male, hound cross, about 1 years old) were anesthetized and nerve cuff electrodes 800 (Evergreen Medical, Minneapolis, MN) were placed around the cervical vagus nerve, as illustrated in FIGS. 8A and 8B. The lead body was externalized and protected under a jacket. Supra-threshold electrical pulses (250-1000 μA, 200 μS, 20 Hz, total of 600 stimuli per burst) were applied seven days apart. Blood was drawn about every three days, and the drawn blood was challenged with endotoxin ex vivo (0.1-0.5 ng/mL LPS) for four hours; the serum TNF levels were measured by ELISA to measure the effects on inflammatory cytokines.

As shown on FIG. 9, even a single 60 second stimulation of the vagus nerve resulted in a substantial suppression of TNF for at least 7 days. Thus, the initial effect was very long lasting. Additionally, when the canines were stimulated for a second time, the same second 60 second stimulation following upon the resulted in suppression of endotoxin-induced TNF for an even longer period of time, about 9-12 days. Thus, the anti-inflammatory effect was trainable to provide progressively increased longevity or duration of anti-inflammatory effect with each successive stimulation. Although the data in FIG. 9 are doses that each include a single burst of supra-threshold pulses (e.g., 600 per dose), the number of pulses may be different, and may be single-pulse. Although the onset of the effect of single pulse on markers for an anti-inflammatory (and/or inhibition of inflammation) response, the duration and extent of the effect is typically the same as that seen for multiple pulses (as discussed above for FIGS. 2-6).

These data illustrate an extremely persistent anti-inflammatory effect of vagus nerve stimulation on the blood of a large mammal with just a single stimulation dose. In this example, a single brief period of stimulation of the vagus nerve results in a surprisingly long-lasting effect (e.g., up to 7 days). Importantly, the persistence of this effect may be lengthened by training the inflammatory system through infrequent stimulations, potentially allowing for effective stimulations to be delivered weekly, monthly, every two months, quarterly or even annually. This data strongly suggests that stimulation may be provided extremely infrequently, with long (e.g., >48 h, >7 days) “silent” periods without stimulation applied. Such extremely low duty-cycle stimulation for treating IBD or rheumatoid arthritis and other diseases mediated by the inflammatory pathway may be particularly helpful in implantable systems, allowing extremely long battery life while having unexpectedly robust therapeutic benefits.

In general, these results suggest that the application of even a single brief stimulus (or burst of stimulus) of the vagus nerve may result in a substantial long term reduction in the effects of inflammation. Furthermore, these results suggest that the duration of the anti-inflammatory effect of the single stimulation may be increased by applying subsequent stimulations after a relatively lengthy period of time between stimulations. These results are surprising, given the prior arts tendency to stimulate for much longer times, and important for the design of future devices and methods. In particular, stimulation of the vagus nerve (or other portions of the inflammatory reflex) may be configured to apply extremely low duty-cycle stimulation. As mentioned briefly, this would allow for much smaller, lighter and more efficient implantable stimulation systems.

FIG. 10 illustrates a prophetic example of a dosing regimen that may maximize the progressively longer off times described above. In FIG. 10, a baseline of 0% inhibition of inflammation is shown prior to starting the stimulation from the implanted microstimulator. At t=0 (day 0), the first dose is applied. As mentioned, the first dose maybe a single supra-threshold pulse, or a single burst (e.g., 1 min, 2 min, 5 min) burst of supra-threshold stimulation, followed by an enforced off-time period, when stimulation is not applied. By day 7, the percent inhibition of inflammation has fallen back to nearly 25% inhibition. Thereafter, a second dose is applied (dose 2), driving inhibition back up. In this example, the inhibition following the second extremely low-duty cycle stimulation (does 2 may be the same or different as the electrical stimulation applied by dose 1) is longer-lasting that following the single dose, allowing a longer off-time period before the percent inhibition falls back to nearly 25% again at day 18 (approximately 10-11 days following dose 2). The third stimulation dose is applied, and the percent inhibition again takes even longer (e.g., 14-15 days) to fall back to nearly 25% inhibition of inflammation. Subsequent additional doses may be applied to sustain the inhibition of inflammation, as illustrated for doses 4 and 5 (or more); the off-time duration may continue to be reduced down to a maximum predetermined off time (e.g., 25 days, 28 days, 30 days, etc.).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature. Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein. For example, as used herein, “about” and “approximately” can mean within 5, 10, 15, 20, 25, or 30 percent.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for treating chronic inflammation in a subject, the system comprising:
an implantable microstimulator configured to apply a low duty-cycle stimulation to a vagus nerve; and
a controller adapted to set a dose regimen, wherein the dose regimen comprises a plurality of electrical stimulations to the vagus nerve that are separated by an off period of at least 4 hours, wherein each electrical stimulation comprises less than 100 supra-threshold pulses.
